(19) United States
(12) Patent Application Publication
(10) Pub. No.: US 2005/0176139 A1
(43) Pub. Date: Aug. 11, 2005

(54) Placental stem cell and methods thereof

(76) Inventors: Yao-Chang Chen, Taipei (TW); Lin-Ju Yen, Taipei (TW); Chih-Cheng Chien, Taipei (TW); Hsing-I Huang, Taipei (TW)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/032,153

(22) Filed: Jan. 11, 2005

Related U.S. Application Data
(60) Provisional application No. 60/535,502, filed on Jan. 12, 2004.

(51) Int. Cl. C12N 5/00; C12N 5/08

(52) U.S. Cl. 435/325; 435/366

(57) ABSTRACT

The present invention describes stem cells obtained from post-partum placenta and their methods of obtaining and culturing. The present invention also describes compositions comprising placental stem cells and methods of using placental stem cells.

### RELATED APPLICATION

This application claims the benefit of U.S. Provisional Application No. 60/535,502, filed Jan. 12, 2004, the contents of which are incorporated herein by reference.

### FIELD OF THE INVENTION

The present invention relates to stem cells obtained from the postpartum placenta and their methods of obtaining and culturing. The present invention further relates to compositions comprising placental stem cells and to methods of using placental stem cells.

### BACKGROUND OF THE INVENTION

Stem cells have the potential to develop into many different cell types in the body. Stem cells can theoretically divide without limit to replenish other cells. When a stem cell divides, each new cell has the potential to either remain a stem cell or become another type of cell with a more specialized function, such as a muscle cell, a red blood cell, or a brain cell. Stem cells are often classified as totipotent, pluripotent, and multipotent. A totipotent stem cell has differentiation potential which is total: it gives rise to all the different types of cells in the body, including the germ cells. A fertilized egg cell is an example of a totipotent stem cell. Pluripotent stem cells can give rise to any type of cell in the body except those needed to develop a fetus. Multipotent stem cells can give rise to two or more different cell types but only within a given organ or tissue type. In contrast to stem cells, progenitor cells are unable to self-renew and they give rise to only a few cell types.

The main sources of stem cells are the embryonic stem cells and adult stem cells. Embryonic stem cells are derived from embryos. For research purposes, embryonic stem cells are obtained from embryos that have developed from eggs that have been fertilized in vitro (such as at an in vitro fertilization clinic) and then donated for research purposes with informed consent of the donors. The embryos are typically obtained at four or five days old when they are a hollow microscopic ball of cells called the blastocyst. The blastocyst includes three structures: the trophoblast, which is the layer of cells that surrounds the blastocyst; the blastocoel, which is the hollow cavity inside the blastocyst; and the inner cell mass, which is a group of approximately 30 cells at one end of the blastocoel.

The embryonic stem cells are obtained by isolating the inner cell mass and growing them in vitro. The inner cell mass is usually grown on a layer of feeder cells, which are mouse embryonic fibroblasts that serve as an adherent layer for the inner cell mass and as a source of nutrients. Embryonic stem cells are pluripotent and can become all cell types of the body.

An adult stem cell, or a somatic stem cell, is an undifferentiated cell found among differentiated cells in a tissue or organ. An adult stem cell can renew itself and can differentiate into specialized cell types of the tissue or organ. They are believed to reside in a specific area of each tissue where they may remain quiescent (non-dividing) for many years until they are activated by disease or tissue injury. Adult stem cells are present in very small numbers in each tissue and have been found in various tissues and organ, including the brain, bone marrow, peripheral blood, blood vessels, skeletal muscle, skin, umbilical cord, adipose tissue, amnion, and liver.

Stem cells have gained considerable interest as a treatment for a myriad of diseases, conditions, and disabilities because they provide a renewable source of cells and tissues. Blood-forming stem cells in bone marrow called hematopoietic stem cells (HSCs) are currently the only type of stem cell commonly used. HSCs are used to treat leukemia, lymphoma and several inherited blood disorders. However, other stem cells have considerable potential for treating many other diseases. A number of reports have suggested that certain adult stem cell types have the ability to differentiate into multiple cell types. For example, hematopoietic stem cells may differentiate into brain cells (neurons, oligodendrocytes, and astrocytes) (Hao et al., H. Hematother. Stem Cell Res. 12: 23-32, 2003; Zhao et al., PNAS 100: 2426-2431, 2003; Bonilla et al., Eur. J. Neurosci. 15: 575-582, 2002), skeletal muscle cells (Ferrariet al., Science 279: 1528-1530, 1998; Gussoni et al., Nature 401: 390-394, 1999), cardiac muscle cells (Jackson et al., J. Clin. Invest. 107: 1395-1402, 2001), and liver cells (Lagasse et al., Nat. Med. 6: 1229-1234, 2000). Bone marrow stromal cells may differentiate into cardiac muscle cells and skeletal muscle cells (Galmiche et al., Blood 82: 66-76, 1993; Wakitani et al., Muscle Nerve 18: 1417-1426, 1995), while brain stem cells may differentiate into blood cells (Bjornson et al., Science 283: 534-547, 1999) and skeletal muscle cells (Galli et al., Nat. Neurosci. 3: 986-991, 2000).

Embryonic and adult stem cells each have advantages and disadvantages regarding potential use for cell-based regenerative therapies. An advantage of adult stem cells is that the patient's own cells may be expanded in culture and reintroduced into the patient. The use of the patient's own adult stem cells would prevent rejection of the cells by the immune system without having to use immunosuppressive drugs. In contrast, embryonic stem cells from a donor introduced into a patient could cause transplant rejection.

Conversely, embryonic stem cells can become all cell types of the body while adult stem cells are generally limited to differentiating into cell types of their tissue of origin, although, as discussed above, some evidence suggests that adult stem cell may differentiate into other cell types. Additionally, relatively large numbers of embryonic stem cells may be grown in culture, while adult stem cells are more rare in adult tissues and it is difficult to expand their numbers in cell culture. In this respect, embryonic stem cells are more advantageous because large numbers of cells are usually needed for stem cell replacement therapies.

However, the use of embryonic stem cells is controversial because of its implications on life. Embryonic stem cells are often obtained from supernumerary embryos from in vitro fertilization programs or from donated gametes. In contrast, adult stem cells pose no ethical dilemma, but their proliferative and differentiation capacity are less than those of embryonic stem cells. Moreover, invasive procedures are usually required to obtain adult stem cells. In addition, embryonic stem cells can cause teratoma formation, a benign tumor consisting of all three germ layers, whereas adult stem cells do not.

### SUMMARY OF THE INVENTION

The present invention provides stem cells from the post-partum placenta. One aspect of the invention provides a method for obtaining a placental stem cell comprising: obtaining a post-partum placenta; preparing a single-cell suspension of placental cells; culturing the placental cells; and obtaining a placental stem cell. The placental stem cell may be multipotent or pluripotent and the placenta may be human placenta.

Another aspect of the invention provides a method for culturing a placental stem cell comprising: obtaining a post-partum placenta; preparing a single-cell suspension of placental cells; culturing the placental cells; obtaining a placental stem cell; and culturing the placental stem cell. The placental stem cell may be multipotent or pluripotent and the placenta may be human placenta.

A further aspect of the present invention provides an isolated placental stem cell having certain characteristics, including cell markers. Another aspect of the invention provides an isolated, homogeneous population of multipotent or pluripotent placental stem cells having certain characteristics, including cell markers.

Thus, an aspect of the invention also provides a method for obtaining a placental stem cell based on certain cell marker characteristics.

Yet, another aspect of the invention provides cryopreserved placental stem cells obtained from a post-partum placenta.

Other aspects of the invention provide a composition comprising a placental stem cell and a pharmaceutical composition comprising a placental stem cell. The invention also provides a method of treating a patient comprising administering to the patient an effective amount of a placental stem cell.

### BRIEF DESCRIPTION OF THE DRAWINGS

FIG. 1 FIG. 1 shows the staining of control bone marrow mesenchymal stem cells as a positive control for Oil Red staining, which detects adipocytic differentiation in red (FIG. 1A), and von Kossa staining, which detects osteogenic differentiation in brown (FIG. 1B).

FIG. 2 FIG. 2 shows the staining of placental stem cells of the invention to demonstrate their differentiation potential into other cell types. FIG. 2A shows unstained placental stem cells and FIG. 2B shows the cells stained with Oil Red to detect adipocytes (in red). FIG. 2C shows the cells stained with alizarin red stain and FIG. 2D shows the cells stained with von Kossa stain, both of which detected osteoblasts (in red and brown, respectively) in the cultured placental stem cells.

FIG. 3 FIG. 3 demonstrates the chondrogenic potential of the placental stem cells of the invention. FIG. 3A shows a placental stem cell-derived cell that stained blue with Alcian Blue dye, indicating that the cell produced proteoglycans. FIG. 3B shows a placental stem cell-derived cell that reacted with anti-Type 11 collagen antibody.

### DESCRIPTION OF THE EMBODIMENTS

The present invention provides stem cells from post-partum placenta. The placenta provides a rich source of stem cells and growth factors because it is of fetal origin and derived from the embryo. Therefore, placental stem cells may be more likely to have a higher proliferative and differentiation capacity than other adult stem cells. Moreover, the placenta is a temporary organ used to ensure fetal survival in utero and is discarded after birth. Placenta obtained post-partum therefore poses no ethical controversy and no invasive procedure is required for procurement of the cells.

The embodiments described and the terminology used herein are for the purpose of describing exemplary embodiments only, and are not intended to be limiting. The scope of the present invention is intended to encompass additional embodiments not specifically described herein, but that would be apparent to one skilled in the art upon reading the present disclosure and practicing the invention.

The present invention relates to stem cells from the post-partum placenta. As used herein, the term “stem cell” refers to a master cell that can reproduce indefinitely to form the specialized cells of tissues and organs. A stem cell can divide to produce two daughter stem cells, or one daughter stem cell and one progenitor (“transit”) cell, which then proliferates into the tissue's mature, fully formed cells. As used herein, the term “stem cell” includes multipotent and pluripotent stem cells.

As used herein, the term “pluripotent cell” refers to a cell that has complete differentiation versatility, i.e., the capacity to grow into any of the mammalian body's cell types, except those needed to develop a fetus. A pluripotent cell can be self-renewing, and can remain dormant or quiescent within a tissue.

As used herein, the term “multipotent cell” refers to a cell that has the capacity to grow into two or more different cell types of the mammalian body within a given tissue or organ. However, a multipotent cell may have the capacity to be pluripotent. For example, hematopoietic stem cells were originally believed to be multipotent cells, i.e., stem cells that could develop into several types of blood cells, but not into brain cells. However, as discussed above, recent evidence suggests that hematopoietic stem cells may be pluripotent because they may differentiate into other types of cells, including brain cells.

As used herein, the term “progenitor cell” refers to a cell that is committed to differentiate into a specific type of cell or to form a specific type of tissue.

As used herein, the term “post-partum placenta” refers to placenta that has been expunged from the uterus after birth and does not include the umbilical cord. Thus, the method of the present invention for obtaining placental stem cells contrasts from prior methods utilizing umbilical cord blood (Migliaccio et al., Blood 96: 2717-2722, 2000; Rubinstein et al., New England J Medicine 339: 1565-1577, 1998; Hariri et al., U.S. Patent Publication No.: 20030180269) or the umbilical cord itself. The placenta may be obtained from any mammalian species, including rodents, human, non-human primates, equines, canines, felines, bovines, porcines, ovines, lagomorphs, and the like. In an embodiment of the invention, the placenta is obtained from human.

A placental stem cell may be characterized by its cell markers. A variety of cell markers are known. See e.g., Stem Cells: Scientific Progress and Future Research Directions. Department of Health and Human Services. June 2001. http://www.nih.gov/news/stemcell/scireport.htm. Cell markers may be detected by methods known in the art, such as by immunochemistry or flow cytometry. Flow cytometry allows the rapid measurement of light scatter and fluorescence emission produced by suitably illuminated cells or particles. The cells or particles produce signals when they pass individually through a beam of light. Each particle or cell is measured separately and the output represents cumulative individual cytometric characteristics. Antibodies specific to a cell marker may be labeled with a fluorochrome so that it may be detected by the flow cytometer. See, eg., Bonner et al., Rev. Sci. Instrum 43: 404-409, 1972; Herzenberg et al., Immunol. Today 21: 383-390, 2000; Julius et al., PNAS 69: 1934-1938, 1972; Ormerod (ed.), Flow Cytometry: A Practical Approach, Oxford Univ. Press, 1997; Jaroszeski et al. (eds.), Flow Cytometry Protocols in Methods in Molecular Biology No. 91, Humana Press, 1997; Practical Flow Cytometry, 3^{rd} ed., Wiley-Liss, 1995.

In an embodiment of the invention, a human placental stem cell expresses at least one of the following cell markers: CD9, CD13, CD29, CD44, CD90/Thy-1, CD105/SH-2/endoglin, CD166, SH-3, SH-4, vimentin, HLA-ABC, SSEA-4, TRA-1-60, and TRA-1-81. In a further embodiment, a human placental stem cell is negative for the at least one of the following cell markers: CD14, CD34, CD45, AC or CD133/2, cytokeratin 7, von Willebrand factor, HLA-DR, HLA G, glycophorin A, placental alkaline phosphatase, and β-human chorionic gonadotropin. In another embodiment of the invention, a placental stem cell is positive for at least CD9, CD13, CD29, CD44, CD90/Thy-1, CD105/SH-2/endoglin, CD166, SH-3, SH-4, vimentin, HLA-ABC, SSEA-4, TRA-1-60, and TRA-1-81, and negative for at least CD14, CD34, CD45, AC or CD133/2, cytokeratin 7, von Willebrand factor, HLA-DR, HLA G, glycophorin A, placental alkaline phosphatase, and β-human chorionic gonadotropin.

The present invention also embodies a homogeneous population of placental stem cells. As used herein, “homogeneous population” refers to a population of cells exhibiting substantially the same phenotype, such as that determined by cell markers. A homogeneous population may comprise at least about 70% of substantially the same cells, or at least about 80%, 90%, 92%, 96%, or 99% of substantially the same cells.

The present invention therefore provides a method of obtaining a placental stem cell by isolating placental cells having certain cell characteristics. The placental cells having these cell characteristics may be isolated from the single-cell suspension of placental cells obtained from post-partum placenta as described above or from placental cells that have been cultured after isolating from the placenta. Cells may be isolated according to cell characteristics by, for example, flow cytometry, as described above. In an embodiment of the present invention, placental stem cells are isolated by isolating placental cells having at least one of the following characteristics:
a. positive for cell markers CD9, CD13, CD29, CD44, CD90/Thy-1, CD105/SH-2/endoglin, CD166, SH-3, SH-4, vimentin, HLA-ABC, SSEA-4, TRA-1-60, and TRA-1-81;
b. negative for cell markers CD14, CD34, CD45, AC or CD133/2, cytokeratin 7, von Willebrand factor, HLA-DR, HLA G, glycophorin A, placental alkaline phosphatase, and β-human chorionic gonadotropin; or
c. positive for cell markers CD9, CD13, CD29, CD44, CD90/Thy-1, CD105/SH-2/endoglin, CD166, SH-3, SH-4, vimentin, HLA-ABC, SSEA-4, TRA-1-60, and TRA-1-81, and negative for cell markers CD14, CD34, CD45, AC or CD133/2, cytokeratin 7, von Willebrand factor, HLA-DR, HLA G, glycophorin A, placental alkaline phosphatase, and β-human chorionic gonadotropin.

The present invention also provides a method for obtaining a placental stem cell. The method comprises obtaining a post-partum placenta, preparing a single-cell suspension of placental cells, culturing the placental cells, and obtaining a placental stem cell. Post-partum placenta may be obtained, for example, with informed consent from a caesarian procedure or normal birth. The placenta may be mechanically cut into smaller pieces of tissue, for example, with scissors. A single-cell suspension may be prepared by enzymatically digesting the placenta with, for example, trypsin, chymotrypsin, lysozyme, amylase, or protease K. The placental cells thus obtained may be cultured in culture medium comprising standard medium, such as DMEM (Gibco) and 10% fetal bovine serum (selected lots, Hyclone), and may be supplemented with glucose and/or antibiotics, as appropriate. Placental stem cells may be obtained by continued culture of the placental cells in the culture medium.

The presence of placental stem cells in culture may be detected by their ability to differentiate into different cell types. For example, the cultured cells may be tested for their ability to undergo adipogenic and/or osteogenic differentiation. Adipocytes are connective tissue cells responsible for the synthesis and storage of fat, while osteoblasts are the primary cells responsible for bone formation and are thought to originate from osteoprogenitor cells within skeletal tissues.

Adipogenic differentiation may be induced in vitro by culturing the cells in 20% rabbit serum, a known inducer of adipogenesis in marrow stromal osteoprogenitor cells (Diascro et al., J. Bone Miner. Res. 13: 96-106, 1988). Adipogenic differentiation may be detected by testing for the presence of adipogenic transcription factors PPARγ2 (peroxisome proliferator activated receptor gamma) and/or CEBPα (CCAAT/enhancer binding protein alpha), by methods such as immunohistochemistry and reverse-transcriptase polymerase chain reaction. Alternatively, adipogenic differentiation may be detected by lipid accumulation as demonstrated by Oil Red O staining after culture in an adipocyte-inducing medium (Conget and Minguell, J. Cellular Physiology 181: 67-73, 1999). Other methods of inducing and detecting adipogenic differentiation may be used (see, e.g., Pittenger et al., Science 284: 143-147, 1999; Tchoukalova et al., Obesity Research 8: 664-672, 2000).

Osteogenic differentiation may be induced by culturing the cells in medium containing, for example, methylisobutylxanthine, dexamethasone, and insulin (Student et al., J. Biol. Chem. 255: 4745-4750, 1980). Osteogenic differentiation may be detected by testing for the presence of osteogenic markers, which include, but are not limited to, osteopontin (OP), osteocalcin (OC), osteonectin (ON), and bone sialoprotein. Osteogenesis may also be detected by using von Kossa stain (Jaiswal et al., J Cell Biochem. 64: 295-312, 1997) and/or alizarin red stain (Wan et al., Chin. J. Traumatol. 5: 374-379, 2002), which detect the presence of calcium deposit activity.

Of course, the placental stem cells of the present invention may be induced into other cell types by methods known in the art.

The present invention also provides a method for culturing a placental stem cell comprising obtaining a post-partum placenta, preparing a single-cell suspension of placental cells, culturing the placental cells, obtaining a placental stem cell, and culturing the placental stem cell. The placental stem cell may be cultured in the same culture medium as that used to culture the single-cell suspension of placental cells.

The present invention further provides a composition comprising a placental stem cell of the invention. The present invention also provides a pharmaceutical composition comprising a placental stem cell of the invention. The placental stem cell of the invention or formulations thereof may be administered by any conventional method including parenteral (e.g. subcutaneous or intramuscular) injection or intravenous infusion. The treatment may consist of a single dose or a plurality of doses over a period of time. The pharmaceutical composition may comprise one or more acceptable carriers. The carrier(s) must be “acceptable” in the sense of being compatible with the placental stem cell and not deleterious to the recipients thereof. Typically, the carriers may be water or saline which will be sterile and pyrogen free.

The placental stem cells of the invention may also be cryopreserved. The cells may be cryopreserved in a solution comprising, for example, dimethyl sulfoxide at a final concentration not exceeding 10%. The cells may also be cryopreserved in a solution comprising dimethyl sulfoxide and/or dextran. Other methods of cryopreserving cells are known in the art.

The present invention provides a method of treating a patient, which comprises administering to the patient a therapeutically effective amount of the placental stem cell of the invention. “Therapeutically effective amount” as used herein, refers to that amount of placental stem cell that is sufficient to reduce the symptoms of the disorder, or an amount that is sufficient to maintain or increase in the patient the number of cells derived from the placental stem cell.

A patient is hereby defined as any person or non-human animal in need of treatment with a placental stem cell, or to any subject for whom treatment may be beneficial, including humans and non-human animals. Such non-human animals to be treated include all domesticated and feral mammals. In an embodiment of the present invention, the placental stem cell to be administered is obtained from the same species as the species receiving treatment. Examples of mammalian species include rodents, human, non-human primates, equines, canines, felines, bovines, porcines, ovines, lagomorphs, and the like.

The placental stem cells of the invention may be used in the treatment of any kind of injury due to trauma where tissues need to be replaced or regenerated. Examples of such trauma-related conditions include central nervous system (CNS) injuries, including injuries to the brain, spinal cord, or tissue surrounding the CNS injuries to the peripheral nervous system (PNS), or injuries to any other part of the body. Such trauma may be caused by accident, or may be a normal or abnormal outcome of a medical procedure such as surgery or angioplasty. The trauma may be related to a rupture or occlusion of a blood vessel, for example, in stroke or phlebitis. In specific embodiments, the cells may be used in autologous or heterologous tissue replacement or regeneration therapies or protocols, including, but not limited to treatment of corneal epithelial defects, cartilage repair, facial dermabrasion, mucosal membranes, tympanic membranes, intestinal linings, neurological structures (e.g., retina, auditory neurons in basilar membrane, olfactory neurons in olfactory epithelium), burn and wound repair for traumatic injuries of the skin, or for reconstruction of other damaged or diseased organs or tissues. Injuries may be due to specific conditions and disorders including, but not limited to, myocardial infarction, seizure disorder, multiple sclerosis, stroke, hypotension, cardiac arrest, ischemia, inflammation, age-related loss of cognitive function, radiation damage, cerebral palsy, neurodegenerative disease, Alzheimer's disease, Parkinson's disease, Leigh disease, AIDS dementia, memory loss, amyotrophic lateral sclerosis (ALS), ischemic renal disease, brain or spinal cord trauma, heart-lung bypass, glaucoma, retinal ischemia, retinal trauma, inborn errors of metabolism, adrenoleukodystrophy, cystic fibrosis, glycogen storage disease, hypothyroidism, sickle cell anemia, Pearson syndrome, Pompe's disease, phenylketonuria (PKU), porphyrias, maple syrup urine disease, homocystinuria, mucoplysaccharide nosis, chronic granulomatous disease and tyrosinemia, Tay-Sachs disease, cancer, tumors or other pathological or neoplastic conditions.

The placental stem cell used in the treatment may also contain a nucleic acid vector or biological vector in an amount sufficient to direct the expression of a desired gene(s) in a patient. The construction and expression of conventional recombinant nucleic acid vectors is well known in the art and includes those techniques contained in Sambrook et al., Molecular Cloning: A Laboratory Manual, Vols 1-3 (2d ed. 1989), Cold Spring Harbor Laboratory Press. Such nucleic acid vectors may be contained in a biological vector such as viruses and bacteria, preferably in a non-pathogenic or attenuated microorganism, including attenuated viruses, bacteria, parasites, and virus-like particles.

The nucleic acid vector or biological vector may be introduced into the cells by an ex vivo gene therapy protocol, which comprises excising cells or tissues from a patient, introducing the nucleic acid vector or biological vector into the excised cells or tissues, and reimplanting the cells or tissues into the patient (see, for example, Knoell et al., Am. J. Health Syst. Pharm. 55: 899-904, 1998; Raymon et al., Exp. Neurol. 144: 82-91, 1997; Culver et al., Hum. Gene Ther. 1: 399-410, 1990; Kasid et al., Proc. Natl. Acad. Sci. U.S.A. 87: 473-477, 1990). The nucleic acid vector or biological vector may be introduced into excised cells or tissues by, for example, calcium phosphate-mediated transfection (Wigler et al., Cell 14: 725, 1978; Corsaro and Pearson, Somatic Cell Genetics 7: 603, 1981; Graham and Van der Eb, Virology 52: 456, 1973). Other techniques for introducing nucleic acid vectors into host cells, such as electroporation (Neumann et al., EMBO J. 1: 841-845, 1982), may also be used.

The cells of the invention may also be co-administered with other agents, such as other cell types, growth factors, and antibiotics. Other agents may be determined by those of ordinary skill in the art.

Other than in the operating examples, or where otherwise indicated, all numbers expressing quantities of ingredients, reaction conditions, and so forth used in this application are to be understood as being modified in all instances by the term “about.” Accordingly, unless the contrary is indicated, the numerical parameters set forth in this application are approximations that may vary depending upon the desired properties sought to be obtained by the present invention. At the very least, and not as an attempt to limit the application of the doctrine of equivalents to the scope of the claims, each numerical parameter should be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements.

It must be noted that as used herein and in the appended claims, the singular forms “a”, “and”, and “the” include plural referents unless the context clearly dictates otherwise.

Unless defined otherwise, all technical and scientific terms used herein have the same meaning as commonly understood by one of ordinary skill in the art to which this invention belongs. Although any methods and materials similar or equivalent to those described herein can also be used in the practice of the present invention, exemplary methods and materials are described for illustrative purposes.

All publications mentioned in this application are incorporated by reference to disclose and describe the methods and/or materials in connection with which the publications are cited. Additionally, the publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

Methods, techniques, and/or protocols (collectively “methods”) that can be used in the practice of the invention are not limited to the particular examples of these procedures cited throughout the specification but embrace any procedure known in the art for the same purpose. Furthermore, although some methods may be described in a particular context in the specification, their use in the instant invention is not limited to that context.

The present invention is illustrated by the following Examples, which are not intended to be limiting in any way.

### EXAMPLE 1

### Isolation and Culture of Placental Stem Cell

Term (38-40 wk gestation) placentas from healthy donor mothers were obtained by caesarian section or natural birth with informed consent, which permitted the use of the placenta for research purposes according to the procedures approved by the institutional review board. The placentas were carefully dissected and washed several times in phosphate-buffered saline (PBS). The harvested pieces of tissue were further cut into smaller pieces with scissors and enzymatically digested with 0.25% trypsin-EDTA (Gibco) for 10 minutes at 37° C. The cells were pelleted by centrifugation, washed once with PBS, and suspended in DMEM (Gibco) medium supplemented with 10% FBS (Hyclone), 100 U/ml penicillin (Gibco), and 100 g/ml streptomycin (Gibco). Cell cultures were maintained at 37° C. and 5% CO₂ and in a water-saturated atmosphere. Medium was replaced twice every week. When the plates became more than 80% confluent, the cells were recovered with 0.25% trypsin-EDTA and replated at a ratio of 1:2 to 1:3. Cells were grown for at least 9 days and analyzed.

### EXAMPLE 2

### Adipocytic and Osteogenic Differentiation of Placental Stem Cells

Cells obtained according to Example 1 were cultured either in an adipogenic medium (0.5 mM isobutyl-methylxanthine, 1 μM dexamethasone, 10 μM insulin, and 60 μM indomethacin) (Dennis et al., J. Bone and Mineral Research 14: 700-709, 1999) or osteogenic medium (0.1 μM dexamethasone, 10 mM α-glycerol phosphate and 50 μm ascorbate) (Jaiswal et al., J Cell Biochem. 64: 295-312, 1997). The presence of adipocytes was assessed by the cellular accumulation of neutral lipid vacuoles that stained with Oil Red stain (Conget and Minguell, J. Cellular Physiology 181: 67-73, 1999). Osteoblastic differentiation was evaluated by calcium accumulation with von Kossa stain (Jaiswal et al., J Cell Biochem. 64: 295-312, 1997) or alizarin red stain (Wan et al., Chin. J. Traumatol. 5: 374-379, 2002).

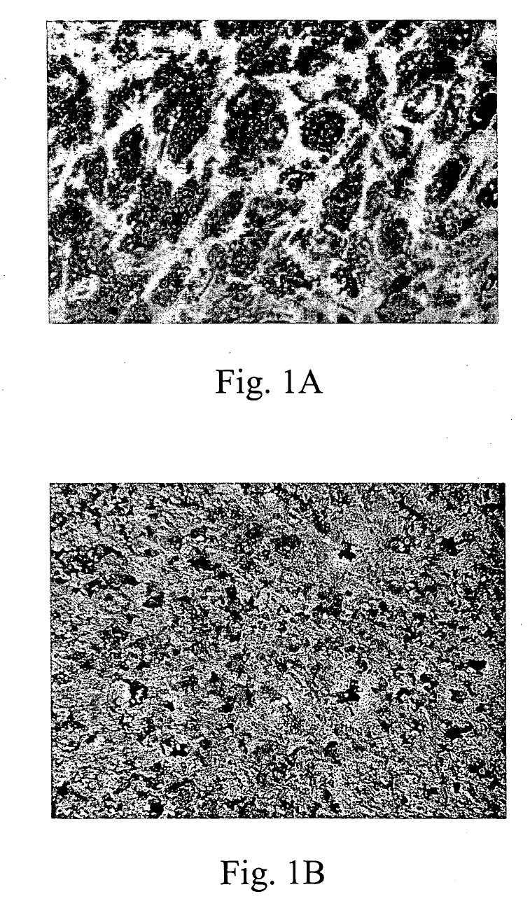

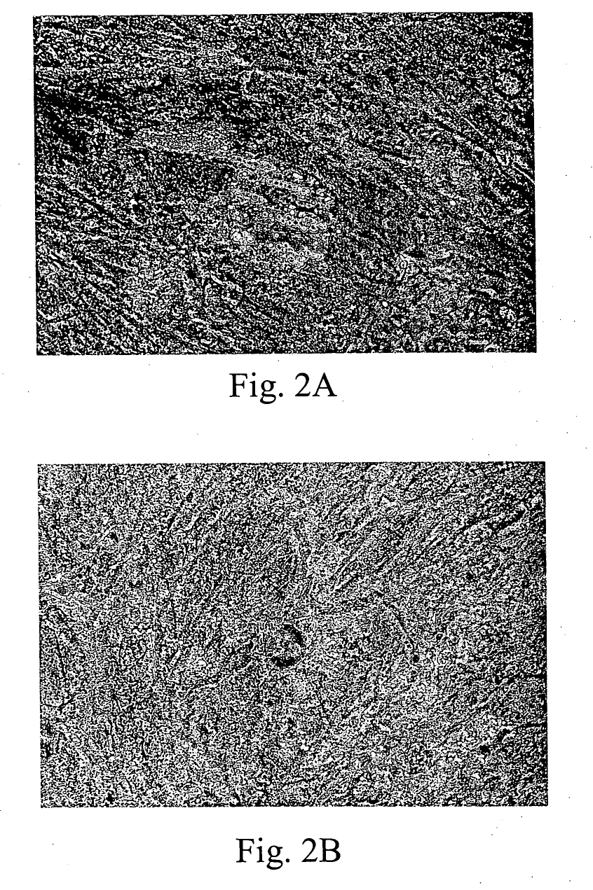

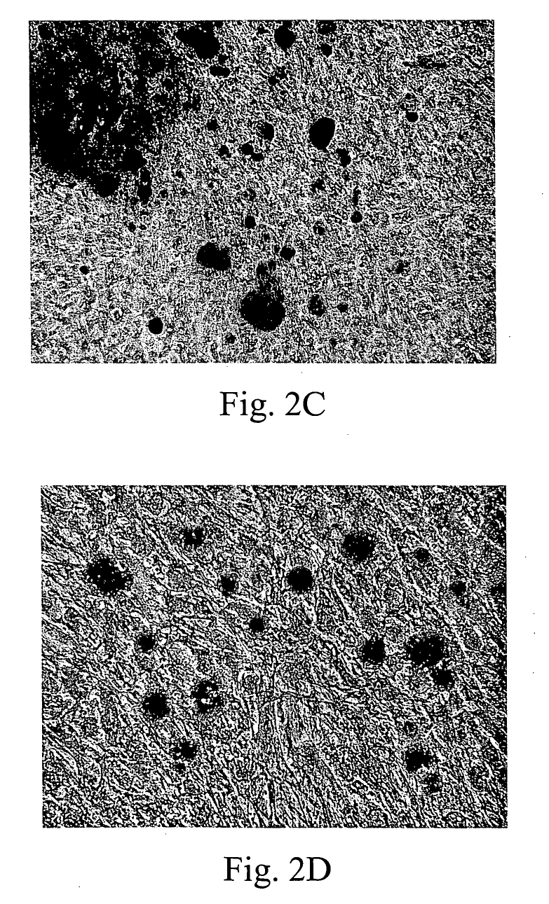

FIG. 1 shows the positive control bone marrow mesenchymal stem cells stained with Oil Red stain to detect adipocytic differentiation (FIG. 1A), and von Kossa stain to detect osteogenic differentiation (FIG. 1B). FIG. 2 demonstrates the differentiation potential of the placental stem cells of the invention. FIG. 2A shows the unstained placental stem cells after adipocytic differentiation, and FIG. 2B shows the placental stem cells after adipocytic differentiation stained with Oil Red to detect intracellular lipid accumulations. FIG. 2B shows that the placental stem cells obtained according to the method of the invention are able to differentiate into adipocytes. The placental stem cells of the invention are also able to differentiate into osteoblasts, as indicated by FIGS. 2C and 2D. Both alizarin red staining (FIG. 2C) and von Kossa staining (FIG. 2D) resulted in the staining of osteoblasts from the cultured placental stem cells. Thus, the placental stem cells are able to undergo differentiation into multiple cell types.

### EXAMPLE 3

### Chondrogenic Differentiation of Placental Stem Cells

A chondroblast is a cell that secretes cartilage matrix and becomes a chondrocyte when it is surrounded by matrix. Placental stem cells obtained according to Example 1 above were induced to undergo chondrogenic differentiation using the micromass culture technique of Barry et al., Experimental Cell Research 269: 189-200, 2001 and Zuk et al., Tissue Engineering 7: 211-28, 2001. Briefly, approximately 200,000 cells were placed in a 15 ml conical polypropylene tube for 3 to 6 hours in medium containing 10 ng/ml transforming growth factor (TGF)-β1 or TGF-β3 (both from R&D systems), 0.1 mM ascorbic acid-2-phosphate (Sigma), 1×107 M dexamethasone (Sigma), and 1% insulin-transferrin-sodium selenite media supplement (Sigma). The cells were transferred to 24-well plates and further cultured for two to three weeks. The cells were then stained with Alcian Blue (pH1) (Sigma) for proteoglycans, which are found in cartilage and other connective tissues. Additionally, Type II collagen was detected in the cultured cells by immunocytochemistry using anti-human collagen type II antibodies (Santa Cruz Biotechnology, Inc.) at a 1:100 dilution after overnight incubation at 4° C. Subsequent secondary antibody staining was performed using biotinylated anti-goat IgG secondary antibodies (1:500 dilution, ABC kit, Vector Labs) for 45 minutes at room temperature. Visualization was performed using a Leica DM IRB inverted microscope (Leica, Germany).

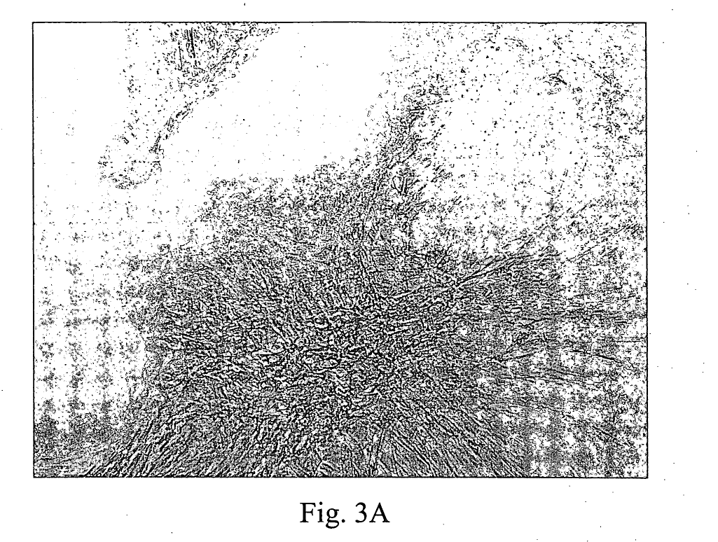

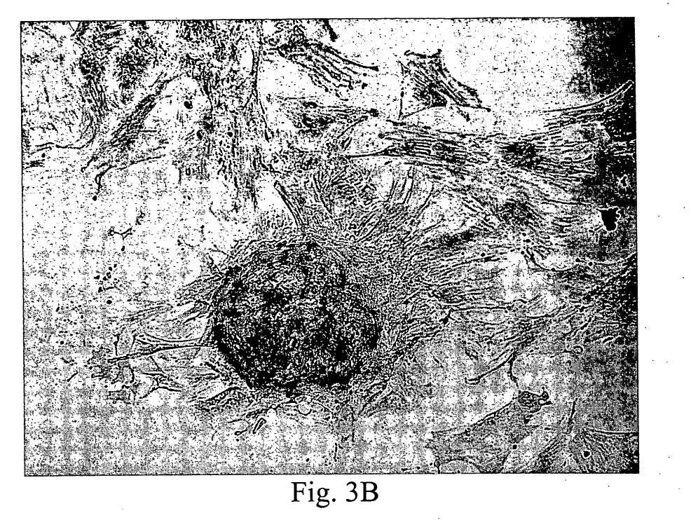

FIGS. 3A and 3B confirm that the placental stem cells of the invention have chondrogenic potential. Specifically, FIG. 3A shows a placental stem cell-derived cell that stained blue with Alcian Blue dye, indicating that the cell produced proteoglycans. FIG. 3B also shows a placental stem cell-derived cell that reacted with anti-Type II collagen antibody, indicating that the cell produced Type II collagen, which is a molecule found mostly in cartilage and which is essential for the normal development of bones and other connective tissues.

### EXAMPLE 4

### Phenotypic Characterization of Placental Stem Cells

The placental stem cells obtained in Example 1 were analyzed for cell markers by flow cytometry and/or immunochemical staining. Table 1 shows a comparison of the cell markers tested on bone marrow (BM) according to Pittenger et al. (Science 284: 143-147, 1999) and Reyes et al. (Blood 98: 2615-1625, 2001), umbilical cord blood (UCB) according to Erices et al. (Br. J. Haematol. 109: 235-242, 2000), and placental stem cells obtained in Example 1 (MPSC).

TABLE 1

| | Pittenger | Reyes | | |
|---|---|---|---|---|
| | et al | et al | Erices | |
| | (Bone | (Bone | et al | |
| Cell Markers | marrow) | marrow) | (UCB) | MPSC |
| CD9 | (+) | | | (+) |
| CD13 | | (+) | (+) | (+) |
| CD14 | (−) | | (−) | (−) |
| CD29 | (+) | | (+) | (+) |
| CD34 | (−) | (−) | (−) | (−) |
| CD44 | (+) | (+) | | (+) |
| CD45 | (−) | (−) | (−) | (−) |
| CD90/Thy-1 | (+) | | (+) | (+) |
| CD105/SH2/ | (+) | | | (+) |
| endoglin | | | | |
| CD117/c-kit | | (−) | | (−) |
| AC or CD133/2 | | | | (−) |
| CD166 | (+) | | | (+) |
| SH3 | (+) | | (+) | (+) |
| SH4 | (+) | | (+) | (+) |
| Vimentin | | | | (+) |
| Cytokeratin 7 | | | | (−) |
| von Willebrand | (−) | | (−) | (−) |
| factor | | | | |
| HLA-ABC | (+) | (−) | | (+) |
| HLA-DR | (−) | (−) | | (−) |
| HLA-G | | | | (−) |
| Glycophorin A | | (−) | | (−) |
| SSEA-4 | | | | (+) |
| TRA-1-60 | | | | (+) |
| TRA-1-81 | | | | (+) |
| β-human | | | | (+) |
| chorionic | | | | |
| gonadotropin | | | | |
| Placental alkaline | | | | (−) |
| phosphatse | | | | |

The specification is most thoroughly understood in light of the teachings of the references cited within the specification, all of which are hereby incorporated by reference in their entirety. The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with the true scope and spirit of the invention being indicated by the following claims.

1. A method for obtaining a placental stem cell comprising:
a. obtaining a post-partum placenta;
b. preparing a single-cell suspension of placental cells;
c. culturing the placental cells; and
d. obtaining a placental stem cell.

2. The method of claim 1, wherein the placenta is human placenta.

3. The method of claim 1, wherein the single-cell suspension is prepared by enzymatically digesting the placenta.

4. The method of claim 1, wherein the placental stem cell is multipotent.

5. The method of claim 1, wherein the placental stem cell is pluripotent.

6. A method for culturing a placental stem cell comprising:
a. obtaining a post-partum placenta;
b. preparing a single-cell suspension of placental cells; and
c. culturing the placental cells;
d. obtaining a placental stem cell; and
e. culturing the placental stem cell.

7. The method of claim 6, wherein the placenta is human placenta.

8. The method of claim 6, wherein the single-cell suspension is prepared by enzymatically digesting the placenta.

9. The method of claim 6, wherein the placental stem cell is multipotent.

10. The method of claim 6, wherein the placental stem cell is pluripotent.

11. A multipotent placental stem cell obtained by the method of claim 1.

12. A pluripotent placental stem cell obtained by the method of claim 1.

13. The placental stem cell of claim 11 or 12, wherein the placental stem cell is human placental stem cell.

14. An isolated placental stem cell having at least one of the following characteristics:
a. positive for cell markers CD9, CD13, CD29, CD44, CD90/Thy-1, CD105/SH-2/endoglin, CD166, SH-3, SH-4, vimentin, HLA-ABC, SSEA-4, TRA-1-60, and TRA-1-81;
b. negative for cell markers CD14, CD34, CD45, AC or CD133/2, cytokeratin 7, von Willebrand factor, HLA-DR, HLA G, glycophorin A, placental alkaline phosphatase, and β-human chorionic gonadotropin; or
c. positive for cell markers CD9, CD13, CD29, CD44, CD90/Thy-1, CD105/SH-2/endoglin, CD166, SH-3, SH-4, vimentin, HLA-ABC, SSEA-4, TRA-1-60, and TRA-1-81, and negative for cell markers CD14, CD34, CD45, AC or CD133/2, cytokeratin 7, von Willebrand factor, HLA-DR, HLA G, glycophorin A, placental alkaline phosphatase, and β-human chorionic gonadotropin.

15. The isolated placental stem cell of claim 14, wherein the placental stem cell is multipotent.

16. The isolated placental stem cell of claim 14, wherein the placental stem cell is pluripotent.

17. An isolated, homogeneous population of placental stem cells having at least one of the following characteristics:
a. positive for cell markers CD9, CD13, CD29, CD44, CD90/Thy-1, CD105/SH-2/endoglin, CD166, SH-3, SH-4, vimentin, HLA-ABC, SSEA-4, TRA-1-60, and TRA-1-81;
b. negative for cell markers CD14, CD34, CD45, AC or CD133/2, cytokeratin 7, von Willebrand factor, HLA-DR, HLA-G, glycophorin A, placental alkaline phosphatase, and β-human chorionic gonadotropin; or
c. positive for cell markers CD9, CD13, CD29, CD44, CD90/Thy-1, CD105/SH-2/endoglin, CD166, SH-3, SH-4, vimentin, HLA-ABC, SSEA-4, TRA-1-60, and TRA-1-81, and negative for cell markers CD14, CD34, CD45, AC or CD133/2, cytokeratin 7, von Willebrand factor, HLA-DR, HLA-G, glycophorin A, placental alkaline phosphatase, and β-human chorionic gonadotropin.

18. A composition comprising a placental stem cell of claim 11, 12, 14 or 17.

19. A pharmaceutical composition comprising a placental stem cell of claim 11, 12, 14 or 17.

20. A cryopreserved placental stem cell of claim 11, 12, 14 or 17.

21. A method of treating a patient comprising administering to the patient a therapeutically effective amount of a placental stem cell of claim 11, 12, 14 or 17.

22. A method for obtaining a placental stem cell comprising isolating a placental stem cell having at least one of the following characteristics:
a. positive for cell markers CD9, CD13, CD29, CD44, CD90/Thy-1, CD105/SH-2/endoglin, CD166, SH-3, SH-4, vimentin, HLA-ABC, SSEA-4, TRA-1-60, and TRA-1-81;
b. negative for cell markers CD14, CD34, CD45, AC or CD133/2, cytokeratin 7, von Willebrand factor, HLA-DR, HLA G, glycophorin A, placental alkaline phosphatase, and β-human chorionic gonadotropin; or
c. positive for cell markers CD9, CD13, CD29, CD44, CD90/Thy-1, CD105/SH-2/endoglin, CD166, SH-3, SH-4, vimentin, HLA-ABC, SSEA-4, TRA-1-60, and TRA-1-81, and negative for cell markers CD14, CD34, CD45, AC or CD133/2, cytokeratin 7, von Willebrand factor, HLA-DR, HLA G, glycophorin A, placental alkaline phosphatase, and β-human chorionic gonadotropin.

23. The method of claim 22, wherein the placental stem cell is multipotent.

24. The method of claim 22, wherein the placental stem cell is pluripotent.
